(19) United States
(12) Patent Application Publication
(10) Pub. No.: US 2018/0133100 A1
(43) Pub. Date: May 17, 2018

(54) Massage Device Alleviating Rhinitis

(71) Applicant: Jong Sik Park, Seoul (KR)

(72) Inventor: Jong Sik Park, Seoul (KR)

(21) Appl. No.: 15/578,976

(22) PCT Filed: May 23, 2016
(86) PCT No.: PCT/KR2016/005445

§ 371 Date: Dec. 1, 2017

(30) Foreign Application Priority Data
Jun. 12, 2015 (KR) 10-2015-0083316

(51) Int. Cl. A61H 23/02; A61N 7/00; A61N 5/06; A61N 1/04

(52) U.S. Cl. CPC A61H 23/02; A61N 7/00; A61N 5/0613; A61N 1/0472; A61H 2201/50; A61N 2005/067; A61H 2201/02; A61H 2201/10; A61H 2201/1604; A61N 2007/0073; A61N 2007/0043; A61H 2201/0157

(57) ABSTRACT

The present invention relates to a massage device for alleviating rhinitis, which can alleviate rhinitis symptoms by massaging both sides of the nose using low-frequency waves, ultrasonic waves, high-frequency waves, thermotherapeutic stimulation, or vibration stimulation, and which is fabricated in a compact size and thus is easy to carry and to use.

### TECHNICAL FIELD

The present invention relates generally to a massage device for alleviating rhinitis and, more particularly, to a massage device for alleviating rhinitis, which can alleviate rhinitis symptoms by massaging both sides of the nose using low-frequency waves, ultrasonic waves, high-frequency waves, thermotherapeutic stimulation, or vibration stimulation, and which is fabricated in a compact size and thus is easy to carry and to use.

### BACKGROUND ART

Recently, mortality from various infectious diseases has decreased due to advancement of medical science, whereas incidence of cerebrovascular disease, nervous breakdown, and chronic diseases has increased. The main cause of this is physical disability resulting from stenosis of blood vessels or accumulation of waste products in the blood vessels due to busy life, overwork, accumulation of fatigue, mental tension, irregular eating habits, lack of exercise, etc.

In particular, the blood vessels play an important role in human biology. In young adults, the blood vessels are elastic and blood circulation is ideal. However, as the years go by, harmful deposits accumulate on the inner walls of the blood vessels. As a result, the vessel diameter narrows and the blood vessel walls are highly likely to become hardened, which causes symptoms such as dizziness, paresthesia, lethargy, hypertension, etc.

Meanwhile, a low-frequency therapy device has become popular as a physical therapy device for more effectively stimulating the body part through which the blood vessels passes. In particular, low-frequency electrical stimulation has been used to treat various diseases in various forms, and recently has been combined with acupuncture in oriental medicine to achieve a considerable therapeutic effect. A low-frequency therapy has been reported to be effective on tinnitus in many foreign papers, and has been associated with acupuncture to also achieve a significant therapeutic effect on rhinitis and sinusitis.

A conventional low-frequency therapy device is a device that forcibly exercises muscles to perform necessary therapy, or loosens knotted muscles by applying low-frequency waves to specific parts of the human body, and includes a low-frequency generator, a plurality of pad-type conductive plates, and a cord that are provided as one set. The conductive plate is connected to the low-frequency generator by the cord while being attached to the site to be treated, whereby the therapy is performed.

Meanwhile, among physical abnormalities, an excessive flow of mucus from the nose cannot be completely treated even after long-term therapy, and if it develops into rhinitis, it may be accompanied by discomfort and insomnia. There has been reported research in many academic circles that an effective therapeutic effect can be achieved by low-frequency stimulation.

Rhinitis and sinusitis diseases are widespread regardless of age and sex, and most patients require long-term treatment, but have difficulty in visiting the hospital for the frequent treatments. Although low-frequency electrical stimulation through the low-frequency therapy device has a significant therapeutic effect on the patients with rhinitis and sinusitis, the conventional low-frequency therapy device in the type of an attachment pad is problematic in that it is impossible to treat rhinitis and is inconvenient to use. In other words, since the conventional low-frequency therapy device is attached to the body by the attachment pad, it cannot be easily worn on the nose. When the planar-shaped attachment pad is attached to the nose, a wearer must press the attachment pad by his or her hand, and bring the attachment pad into close contact with the nose using a separate holding mechanism such that the attachment pad is prevented from being separated from the nose, which leads to inconvenience in use.

In an effort to solve such a problem, a transcutaneous electrical stimulation device for rhinitis (hereinafter referred to as a conventional technique) is disclosed in Korean Utility Model Registration No. 20-0370758 (2004 Dec. 8). The conventional technique is configured such that low-frequency electromagnetic waves are generated by a low-frequency generator and the low-frequency electromagnetic waves are delivered to a specific part of the body whereby the body part is treated, and includes: a wearing portion applying low-frequency electricity generated from the low-frequency generator and worn on the face of the body; a holding portion connected to the wearing portion, inducing the low-frequency electricity, and having a hole in which the nose of the body is held; and a plurality of embedded electrodes embedded along the shape of the holding portion and transferring the low-frequency electromagnetic waves generated from the low-frequency generator to the nose.

However, the above-described conventional technique is also problematic in that a wearer must wear the wearing portion on the face, and provision of the low-frequency generator is required, whereby it is inconvenient to carry, store, transfer, and use.

### DISCLOSURE

### Technical Problem

Accordingly, the present invention has been made keeping in mind the above problems occurring in the prior art, and an object of the present invention is to provide a massage device for alleviating rhinitis, which can alleviate rhinitis symptoms by massaging both sides of the nose using low-frequency waves, ultrasonic waves, high-frequency waves, thermotherapeutic stimulation, or vibration stimulation, and which is fabricated in a compact size and thus is easy to carry and to use.

### Technical Solution

In order to accomplish the above object, according to one aspect of the present invention, there is provided a massage device for alleviating rhinitis, the massage device including: a main body having a size and a shape configured to be operable by one hand; massage portions protruding from opposite sides of an upper portion of the main body, respectively, such that the massage portions are placed on opposite sides of a nose, and selectively generating low-frequency waves, ultrasonic waves, high-frequency waves, thermotherapeutic stimulation, and vibration stimulation upon contact with skin; a seat portion concavely provided between the massage portions and resting on a bridge of the nose; and an auxiliary massage portion operatively provided between the massage portions.

In order to accomplish the above object, according to another aspect of the present invention, there is provided a massage device for alleviating rhinitis, the massage device including: a main body having a size and a shape configured to be operable by one hand; massage portions protruding from upper and lower sides of an upper portion of the main body, respectively, such that the massage portions are placed on opposite sides of a nose, and selectively generating low-frequency waves, ultrasonic waves, high-frequency waves, thermotherapeutic stimulation, and vibration stimulation upon contact with skin; a seat portion concavely provided between the massage portions and resting on a bridge of the nose; and an auxiliary massage portion operatively provided between the massage portions.

The seat portion may be made of a flexible synthetic resin material such that the bridge of the nose is smoothly seated in and supports the seat portion.

The auxiliary massage portion may be a lamp from which a laser is emitted, or an electrode from which electrical stimulation is generated.

### Advantageous Effects

As described above, according to the massage device for alleviating rhinitis according to the present invention, it is possible to alleviate rhinitis symptoms by massaging both sides of the nose using low-frequency waves, ultrasonic waves, high-frequency waves, thermotherapeutic stimulation, or vibration stimulation, and to realize a compact size that is easy to carry and use.

### DESCRIPTION OF DRAWINGS

FIG. 1 is a perspective view showing a massage device for alleviating rhinitis according to a preferred embodiment of the present invention.

FIGS. 2a and 2b are views showing a state of use of the massage device for alleviating rhinitis according to the preferred embodiment of the present invention.

FIG. 3 is a perspective view showing a massage device for alleviating rhinitis according to another embodiment of the present invention.

### MODE FOR INVENTION

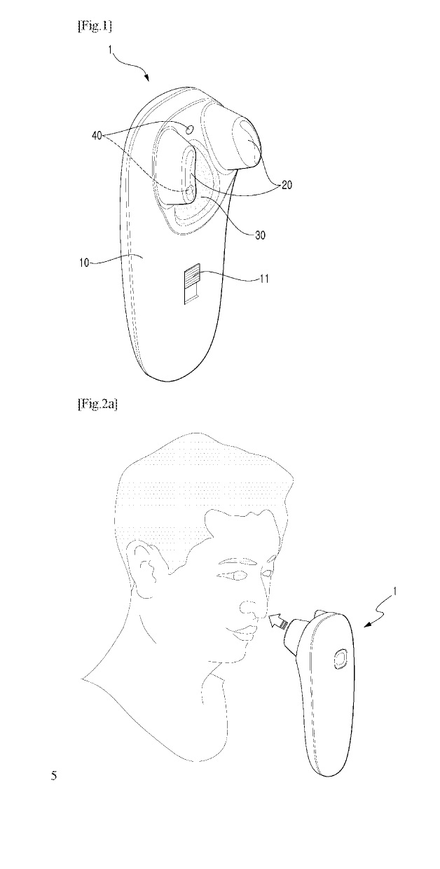

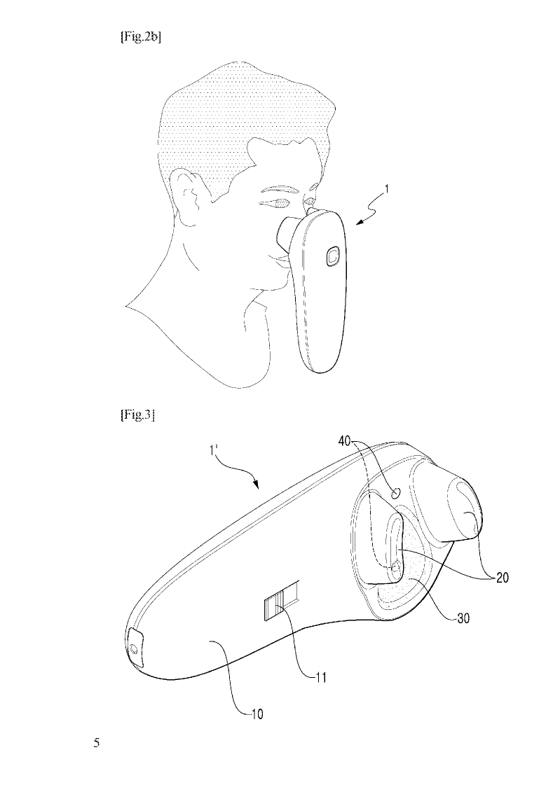

FIG. 1 is a perspective view showing a massage device for alleviating rhinitis according to a preferred embodiment of the present invention, and FIGS. 2a and 2b are views showing a state of use of the massage device for alleviating rhinitis according to the preferred embodiment of the present invention.

The massage device for alleviating rhinitis according to the preferred embodiment of the present invention includes: a main body having a size and a shape configured to be operable by one hand; massage portions protruding from opposite sides of an upper portion of the main body, respectively, such that the massage portions are placed on opposite sides of the nose, the massage portion selectively generating low-frequency waves, ultrasonic waves, high-frequency waves, thermotherapeutic stimulation, and vibration stimulation upon contact with the skin; a seat portion concavely provided between the massage portions and resting on the bridge of the nose; and an auxiliary massage portion operatively provided between the massage portions.

Hereinafter, constituent members, operation relationship thereof, and a method of use of the massage device 1 for alleviating rhinitis according to the preferred embodiment of the present invention will be described in detail with reference to FIGS. 1 to 2b.

Herein, the main body 10 is provided to have a size and a shape configured to be operable by one hand, in which the constituent members that will be described later are operatively installed. In other words, the main body 10 described above is operable by one hand like an automatic razor, and although not shown, a power supply means supplying electric power necessary for the massage portions 20, a low-frequency generating means generating low-frequency waves, and a substrate transmitting and receiving electrical signals necessary for supply of power and operation are respectively operatively installed inside the main body. In addition, a controller 11 controlling operation of the massage portions 20 is provided outside the main body 10.

The power supply means, the low-frequency generating means, and the substrate remain the same as a power supply means, a low-frequency generating means, and a substrate according to the related art, and the power supply means may be a replaceable disposable battery, a rechargeable battery or the like.

The massage portions 20 are provided at the opposite sides of the upper portion of the main body 10 as described above. The massage portions 20 are placed on the opposite sides of the nose, and serve to alleviate rhinitis using low-frequency waves, waves, high-frequency waves, thermotherapeutic stimulation, or vibration stimulation upon contact with the skin.

The massage portions 20 are operated such that the massage portions becomes conductive upon contact with the skin whereby low-frequency waves, ultrasonic waves, high-frequency waves, thermotherapeutic stimulation, or vibration stimulation is automatically generated to stimulate and massage the nose. Each of the massage portions 20 has therein an element capable of generating the corresponding stimulation according to the type of the stimulation. Although the massage portion has a shape protruding to come into close contact with the opposite sides of the nose as shown in the drawing, the massage portion may have a shape that can accommodate the entire nose, and the shape thereof may be variously modified.

The massage portions 20 described above may be operated such that low-frequency stimulation is caused to be generated only when the massage portions become electrically conductive upon contact with the skin although the controller 11 is adjusted to the ON state, or low-frequency stimulation is caused to be generated immediately when the controller 11 is adjusted to the ON state.

Further, the seat portion 30 is concavely provided between the massage portions 20. The seat portion serves to receive therein the bridge of the nose protruding from the face, thereby holding the position of the device on the face. The seat portion extends downward between the massage portions 20, and is made of a flexible synthetic resin material such that the bridge of the nose is allowed to be smoothly seated in and supports the seat portion.

In other words, the protruding bridge of the nose may be pressed into the seat portion 30 while the opposite sides of the nose are massaged by the massage portions 20. Accordingly, as the seat portion 30 is made of a soft and flexible synthetic resin material, even when the massage time is prolonged, it is possible to prevent the wearer's nose from being excessively pressed and to prevent pain from occurring thereon.

In addition, the auxiliary massage portion 40 is operatively provided between the massage portions 20. The auxiliary massage portion 40 serves to exert a skin-improving effect such as minimization of pores of the skin of the nose, skin regeneration, and the like. Two auxiliary massage portions 40 are provided such that one of the auxiliary massage portions is provided between the massage portions 20 and a remaining one is provided in the seat portion 30. The auxiliary massage portion is a lamp from which a laser is emitted or an electrode from which electrical stimulation is generated.

FIG. 3 is a perspective view showing a massage device for alleviating rhinitis according to another embodiment of the present invention. Here, it is to be noted that the same reference numerals are used for the same components as the massage device 1 for alleviating rhinitis according to the preferred embodiment of the present invention.

According to another embodiment of the present invention, the massage device 1′ for alleviating rhinitis includes: a main body 10 having a size and a shape configured to be operable by one hand, and massage portions 20 protruding from upper and lower sides of an upper portion of the main body 10, respectively, such that the massage portions are placed on opposite sides of the nose, and selectively generating low-frequency waves, ultrasonic waves, high-frequency waves, thermotherapeutic stimulation, and vibration stimulation upon contact with the skin; and a seat portion 30 concavely provided between the massage portions 20 and resting on the bridge of the nose.

As shown in FIG. 3, the configuration and the operation of the massage device 1′ for alleviating rhinitis according to another embodiment of the present invention remain the same as those of the massage device 1 for alleviating rhinitis according to the preferred embodiment of the present invention, except that the massage portions 20 are positioned at the upper and lower sides of the upper portion of the main body 10 rather than at the left and right sides, and the seat portion 30 is positioned between the massage portions 20 positioned at the upper and lower sides of the upper portion of the main body whereby a wearer uses the main body 10 while operating it in the horizontal direction rather than in the vertical direction. Accordingly, the configuration and the operation of the present invention can be inferred from the above description, so duplicate explanation will be omitted for simplification of the description.

Although a preferred embodiment of the present invention has been described for illustrative purposes, those skilled in the art will appreciate that various modifications, additions and substitutions are possible, without departing from the scope and spirit of the invention as disclosed in the accompanying claims. Therefore, the patent right of the present invention is not limited to the preferred embodiment described above.

### INDUSTRIAL APPLICABILITY

The present invention can be used for alleviating symptoms of rhinitis in a home or a hospital.

1. A massage device for alleviating rhinitis, the device comprising:
a main body having a size and a shape configured to be operable by one hand;
massage portions protruding from both sides of an upper portion of the main body, respectively, such that the massage portions are placed on both sides of a nose, and generating one of low-frequency waves, ultrasonic waves, high-frequency waves, thermotherapeutic stimulation, and vibration stimulation upon contact with skin;
a nose resting portion concavely provided between the massage portions and resting on a bridge of the nose; and
an auxiliary massage portion operatively provided between the massage portions.

2-4. (canceled)

5. The device of claim 1, wherein the seatnose resting portion is made of a flexible synthetic resin material such that the bridge of the nose is smoothly seated in and supports is supported by the seatnose resting portion.

6. The device of claim 5, wherein the auxiliary massage portion is a lamp from which a laser is emitted, or an electrode from which electrical stimulation is generated.

7. A massage device for alleviating rhinitis, the device comprising:
a main body having a size and a shape configured to be operable by one hand;
massage portions protruding from upper and lower sides of an upper portion of the main body, respectively, such that the massage portions are placed on both sides of a nose, and generating one of low-frequency waves, ultrasonic waves, high-frequency waves, thermotherapeutic stimulation, and vibration stimulation upon contact with skin;
a nose resting portion concavely provided between the massage portions and resting on a bridge of the nose; and
an auxiliary massage portion operatively provided between the massage portions.

8. The device of claim 7, wherein the nose resting portion is made of a flexible synthetic resin material such that the bridge of the nose is smoothly seated in and is supported by the nose resting portion.

9. The device of claim 8, wherein the auxiliary massage portion is a lamp from which a laser is emitted, or an electrode from which electrical stimulation is generated.
